(19) United States
(12) Patent Application Publication
(10) Pub. No.: US 2024/0189821 A1
(43) Pub. Date: Jun. 13, 2024

(54) Method and Device for Trapping at Least One Nucleated Cell Using at Least One Electrode for a Microfluidic Device

(71) Applicant: Robert Bosch GmbH, Stuttgart (DE)

(72) Inventors: Thomas Buck, Tamm (DE); Samir Kadic, Langenau (DE); Franz Laermer, Weil Der Stadt (DE); Michael Dreschmann, Muenchen (DE); Jochen Hoffmann, Renningen (DE); Anne Serout, Aidlingen (DE)

(21) Appl. No.: 18/555,167

(22) PCT Filed: Apr. 20, 2022
(86) PCT No.: PCT/EP2022/060330

§ 371 Date: Oct. 12, 2023

(30) Foreign Application Priority Data
Apr. 20, 2021 (DE) 10 2021 203 896.4
Apr. 19, 2022 (DE) 10 2022 203 848.7

(51) Int. Cl. B01L 3/00

(52) U.S. Cl. CPC B01L 3/502761; B01L 3/50273; B01L 2200/0642; B01L 2200/0652; B01L 2200/12; B01L 2300/0645; B01L 2300/0663; B01L 2300/0829; B01L 2400/0424

(57) ABSTRACT

A method for trapping at least one nucleated cell using at least one electrode for a microfluidic device is disclosed. The method includes (i) outputting an application signal that causes a sample liquid comprising the at least one nucleated cell to be applied to a carrier substrate of the microfluidic device, and (ii) providing a current signal to an interface with the at least one electrode in order to generate, at or in a microcavity of the carrier substrate, an electric field configured to trap the at least one nucleated cell as a target cell in the microcavity.

### THE PRIOR ART

The approach proceeds from a method and a device for trapping at least one nucleated cell using at least one electrode for a microfluidic device, and from a microfluidic device according to the type of invention in the independent claims. The object of the present approach is also a computer program.

Circulating tumor cells (CTCs) have emerged in recent years as promising and clinically relevant biomarkers in the management of advanced malignancies. Their identification and characterization in suitable body fluids, e.g. blood or lymphatic fluid, is known as liquid biopsy—one of the important areas of research in modern oncology.

### DISCLOSURE OF THE INVENTION

Against this background, the approach presented herein presents an improved method for trapping at least one nucleated cell using at least one electrode for a microfluidic device, further an improved device using this method, as well as, finally, a corresponding computer program and an improved microfluidic device according to the main claims. By means of the measures presented in the dependent claims, advantageous embodiments of and improvements to the device specified in the independent claim are enabled.

The approach presented herein enables a low-cost microfluidic device, or efficient and automated microfluidic analysis, using a disposable cartridge.

A method for trapping at least one nucleated cell using at least one electrode for a microfluidic device is presented, the method comprising an outputting step and a providing step. In the outputting step, an application signal is output that causes a sample liquid containing the at least one nucleated cell to be applied to a carrier substrate of the microfluidic device. In the providing step, a current signal is provided to an interface to the at least one electrode to generate an electric field at or in a microcavity of the carrier substrate designed to trap the at least one nucleated cell as a target cell in the microcavity.

The method can, e.g., be performed in the microfluidic device as a lab-on-chip cartridge. Advantageously, the method can be performed in an automated manner. Nucleated cells are cells that comprise a nucleus, which contains genetic material. The nucleated cell can be, e.g., a tumor cell or, e.g., a leukocyte. Accordingly, the sample liquid can be, e.g., a blood sample from a patient. The carrier substrate can, e.g., be PCB-based, i.e. arranged on a printed circuit board, and/or can contain, e.g., a silicon material. For example, the microcavities can be honeycombed, round, or, e.g., angular on or in the carrier substrate. The microcavities can, e.g., be designed to receive and trap the sample liquid containing the at least one nucleated cell for advantageous subsequent analysis.

According to one embodiment, the method can comprise a step of changing an amperage before or after the step of outputting to change the electric field, in particular to strengthen or weaken the field. In particular, the electric field between the electrode and a counter-electrode opposite the electrode in or at the microcavity is established and/or varied. The electric field can advantageously generate a dielectrophoresis cage that can advantageously be opened, closed, or shut off as a function of amperage. The “DEP cage” (open, closed, and shut down) is in this context one aspect of two possible aspects and very useful for trapping cells. To be able to sort cells, starting from positions in microcavities, another aspect of the approach presented herein can be used. Depending on the control of the electrodes in the microcavity, a “DEP Levitator” can also be provided, thus enabling cells to be selectively electrically ejected from the cavities. This has a different distribution of electric field strength than a DEP cage.

Further advantageous is an embodiment of the approach presented herein whereby, during the step of providing, the current signal is output to interface with the at least one electrode and with at least one other electrode arranged in an adjacent microcavity such that a different electric field is generated at the at least one other electrode than is generated at the electrode, in particular whereby the field generated at the electric electrode differs in terms of a direction and/or intensity from the electric field generated at the other electrode and/or whereby the other electric field is generated at the other electrode arranged in a microcavity arranged in a common column or a common row with respect to the microcavity with the electrode. Such an embodiment of the approach proposed herein offers the advantage of preventing or at least mitigating crosstalk of an electric signal caused by an electric field at an electrode of a first microcavity into a second, adjacent microcavity. In this way, very similar or identical potentials can be efficiently prevented from occurring, e.g., at electrodes of microcavities arranged in a common row or a common column of a matrix-shaped structure, and thus a cage formed separately for each microcavity can be achieved. In this way, improved separation or confinement of the cell trapped in the respective microcavity can be implemented.

The method can comprise a step of washing the sample liquid using a wash buffer after the step of providing to wash a suspension of the sample liquid out of the microcavity and the overlying volume of the microfluidic chamber. The wash buffer can advantageously be implemented as a liquid that cleans the suspension but does not damage the nucleated cell. Advantageously, the washing step can improve a transparency of the sample liquid so that, e.g., a subsequent analysis can be easier to perform. The washing step should be gentle enough not to flush the trapped nucleated cell(s) out of their trapping positions.

According to one embodiment, during or after the step of providing, a release signal can be provided to the electrode after trapping the nucleated cell to release another nucleated cell from the sample liquid as a non-target cell from the electric field. Advantageously, the enable signal can trigger an enable voltage that causes the electric field to open. Advantageously, the nucleated cell can thereby be isolated from the further nucleated cell.

According to one embodiment, the outputting step can output the application signal that causes a lysate to be applied to the carrier substrate to obtain a cell sediment containing the at least one nucleated cell and a cell suspension of a lysate. Advantageously, the application signal can be output to an interface to a pumping device so that the pumping device can pump the lysate through the microfluidic device.

Further, the method can comprise a step of identifying the nucleated cells from the sample liquid after the step of providing. In particular, the nucleated cells from the cell sediment can be optically detected and additionally or alternatively quantified in the identification step. Advantageously, the sample liquid can be lysed at or prior to the identifying step and subsequently analyzed to identify whether, e.g., tumor cells are present in the lysate. Lysing can also be performed in the identification step as a preliminary step at the beginning of the sequence.

Also, according to a further embodiment, during the step of providing, the nucleated cell or at least one further nucleated cell can be trapped in a trapping plane of the microcavity and/or whereby the release signal releases the cell or at least one further nucleated cell from the sample liquid from the electric field into a transport plane. In this way, a very efficient trapping and subsequent transport of the respective cell types can be achieved.

Further advantageously, one embodiment of the approach presented herein is a method of trapping at least one nucleated cell using at least one electrode for a microfluidic device, the method comprising a step of applying a sample liquid comprising the at least one nucleated cell to a carrier substrate of the microfluidic device. Further, the method comprises a step of generating an electric field at or in a microcavity of the carrier substrate having the at least one electrode designed to trap the at least one nucleated cell as a target cell in the microcavity. This results in the advantages already specified hereinabove.

It is in this context advantageous if the method comprises a step of changing an amperage to change the electric field, in particular to strengthen or weaken it after the step of applying the sample liquid to a carrier substrate of the microfluidic device, or before or after the step of generating the electric field. The electric field is thereby established and/or varied, e.g., between the electrode and a counter-electrode opposite the electrode in or on the microcavity, This results in the advantages already specified hereinabove.

In addition, it is advantageous if a step of washing the sample liquid using a wash buffer is performed after the step of generating the electric field to wash a suspension of the sample liquid out of the microcavity. This results in the advantages already specified hereinabove.

Furthermore, it is advantageous if during or after the step of generating the electric field after the nucleated cell is trapped, the release of another nucleated cell from the sample liquid as a non-target cell from the electric field is performed by the electrode. This results in the advantages already specified hereinabove.

In addition, it is advantageous if during the step of applying the sample liquid, a lysate is applied to the carrier substrate to obtain a cell sediment containing the at least one nucleated cell and a cell suspension of a lysate. This results in the advantages already specified hereinabove. Furthermore, it is advantageous if the step of identifying the nucleated cells from the sample liquid is provided after the step of generating the electric field. This results in the advantages already specified hereinabove.

Also advantageously, during the step of identification, the nucleated cells from a cell sediment are optically detected and/or quantified. This results in the advantages already specified hereinabove.

Furthermore, it is advantageous if, during the step of generating the electric field, the nucleated cell or at least one further nucleated cell is trapped in a trapping plane of the microcavity and/or whereby, upon release, the cell or at least one further nucleated cell is released from the sample liquid from the electric field into a transport plane. This results in the advantages already specified hereinabove.

This method can, e.g., be implemented as software, or hardware, or in a mixed form of software and hardware, e.g. in a control apparatus.

The approach presented herein furthermore provides a device which is designed in order to perform, control, or change in corresponding devices the steps of a variant of a method presented herein. This embodiment of the approach in the form of a device can also achieve the object underlying the approach quickly and efficiently. The device can, e.g., be realized as a control unit or a control apparatus.

For this purpose, the device can comprise at least one computing unit for processing signals or data, at least one storage unit for storing signals or data, at least one interface to a sensor or an actuator for reading sensor signals from the sensor or for outputting data or control signals to the actuator, and/or at least one communication interface for reading or outputting data embedded in a communication protocol. The computing unit can, e.g., be a signal processor, a microcontroller or the like, whereby the memory unit can be a flash memory, an EEPROM, or a magnetic memory unit. The communication interface can be designed to read or output data in a wireless and/or wired manner, whereby a communication interface capable of reading or outputting wired data can, e.g., electrically or optically read said data from a corresponding data transmission line or output said data into a corresponding data transmission line.

In the present context, the term “device” is understood to mean an electrical apparatus that processes sensor signals and outputs control signals and/or data signals as a function thereof. The device can comprise an interface in the form of hardware and/or software. Given a hardware design, the interfaces can, e.g., be part of what is referred to as an ASIC system, which contains various functions of the device. However, it is also possible that the interfaces are separate integrated circuits or consist at least in part of discrete components. Given a software design, the interfaces can be software modules provided on, e.g., a microcontroller in addition to other software modules.

A computer program product or a computer program comprising program code that can be stored on a machine-readable carrier or storage medium, e.g., a semiconductor memory, a hard disk memory, or an optical memory and can be used for performing, implementing and/or controlling the steps of the method according to one of the embodiments described hereinabove is advantageous as well, in particular when the program product or program is executed on a computer or a device.

A microfluidic device for trapping at least one nucleated cell of a sample liquid is further presented, in particular whereby the microfluidic device can designed as a lab-on-chip cartridge. The microfluidic device comprises a carrier substrate for holding the sample liquid, the carrier substrate having at least one microcavity. Further, the microfluidic device comprises at least one electrode arranged at or within the microcavity to generate an electric field designed to trap the nucleated cell within the microcavity.

The microfluidic device can advantageously be used in conjunction with rapid tests. For example, it can be designed as a disposable cartridge. For example, the carrier substrate can be designed as a layered substrate comprising a plurality of layers. For example, the electrode or several electrodes can be arranged to be integrated into the carrier substrate. The electrode can be arranged on or in a cavity floor, or on or in a cavity wall of the microcavity.

According to one embodiment, the carrier substrate can comprise a plurality of microcavities, each having at least one electrode, whereby the microcavities are arranged on the carrier substrate in a matrix-like manner. Advantageously, an electric field can be generated by the electrodes in the microcavities. Multiple target cells can then be trapped in each microcavity, e.g. one target cell per microcavity.

Further, the at least one electrode can be arranged on a cavity floor and additionally or alternatively in a cavity wall of at least one of the microcavities. Advantageously, this can determine a position at which the nucleated cell can be trapped in the microcavity.

According to one embodiment, the at least one electrode in each of the microcavities can be individually controllable. In other words, advantageously, the at least one electrode can always be targeted in the microcavity in which there is actually a nucleated cell. Specifically, there can further be provided a control unit designed to impress an independent voltage on each of the electrodes in the different microcavities. In this way, crosstalk of electric fields over more than one microcavity can be prevented very efficiently, or such an effect can at least be reduced. Avoiding crosstalk enables setting a new desired field distribution at a single microcavity of interest without changing the already prevailing previous field distributions at all other microcavities.

Furthermore, the electrode can be ring-like, point-like and additionally or alternatively layer-like in shape. Advantageously, each of the microcavities can, e.g., comprise at least two electrodes arranged concentrically. One electrode can be arranged in a point shape on the cavity floor and another electrode can be arranged in a ring shape around the electrode, also on the cavity floor. Alternatively, the at least two electrodes can be arranged in layers on top of each other, i.e., integrated into the carrier substrate.

According to one embodiment, the microcavity can comprise at least one further electrode, whereby the electrode and the at least one further electrode can be electrically insulated from each other. Advantageously, the electrodes can be electrically insulated from each other by means of spatial separation or, e.g., by an isolating layer.

A particularly advantageous embodiment is one in which at least one counter-electrode is arranged on the microcavity, the counter-electrode being arranged opposite the electrode and/or the at least one further electrode in or on the microcavity and/or being electrically insulated from the electrode and/or the at least one further electrode. Such an embodiment enables very flexible control of the electric field in the microcavity, so that individual cells in the microcavity can be separated or separated quickly and easily.

Exemplary embodiments of the approach presented herein are shown in the drawings and explained in greater detail in the subsequent description. Shown are:

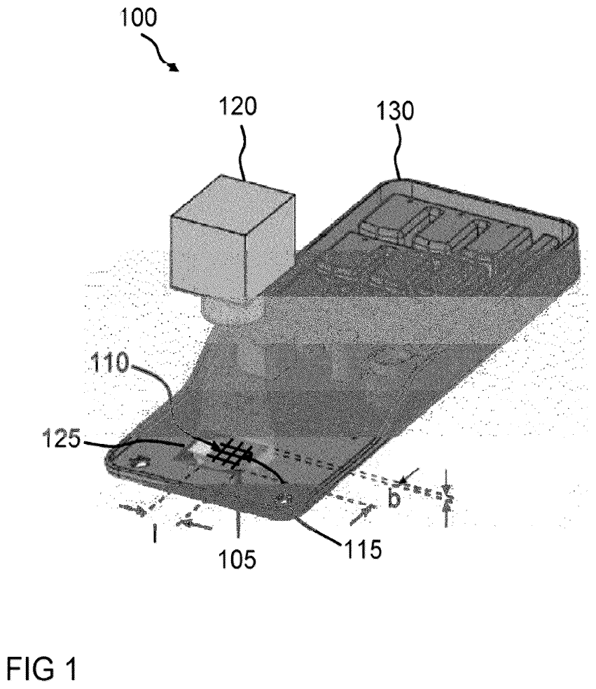

FIG. 1 a schematic illustration of a microfluidic device according to an exemplary embodiment;

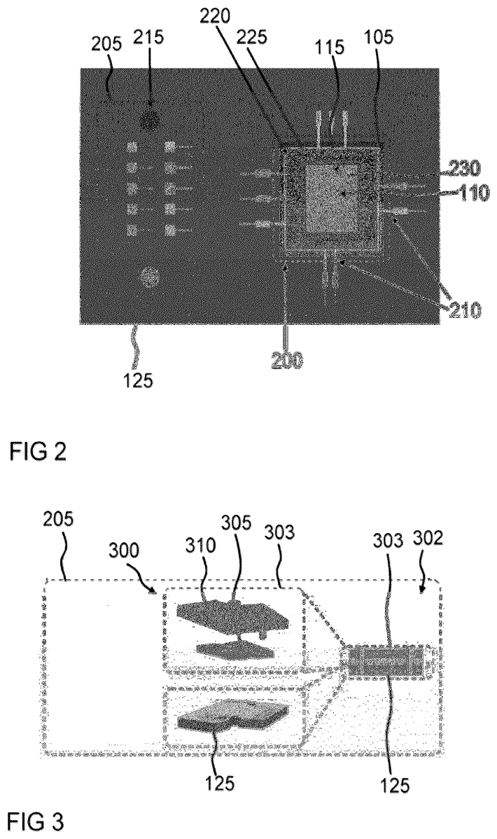

FIG. 2 a schematic illustration of a printed circuit board according to an exemplary embodiment with a carrier substrate;

FIG. 3 a schematic illustration of an interposer region according to an exemplary embodiment;

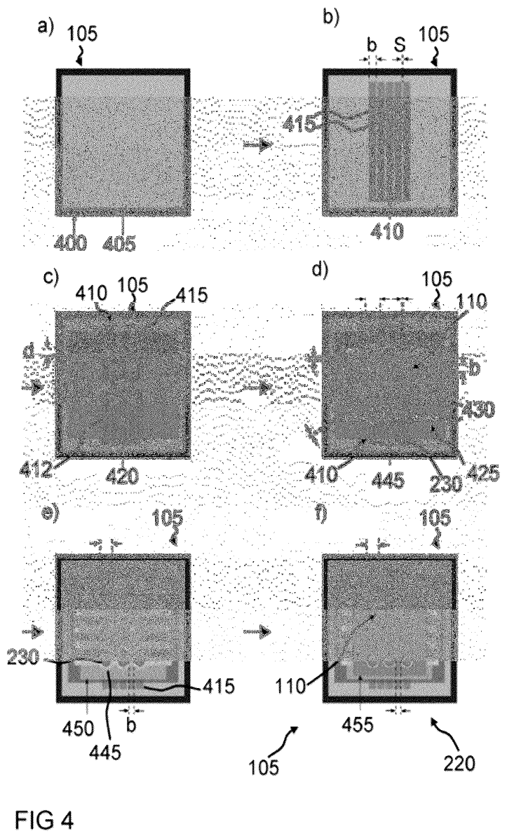

FIG. 4 a schematic assembly diagram of an exemplary embodiment of a carrier substrate;

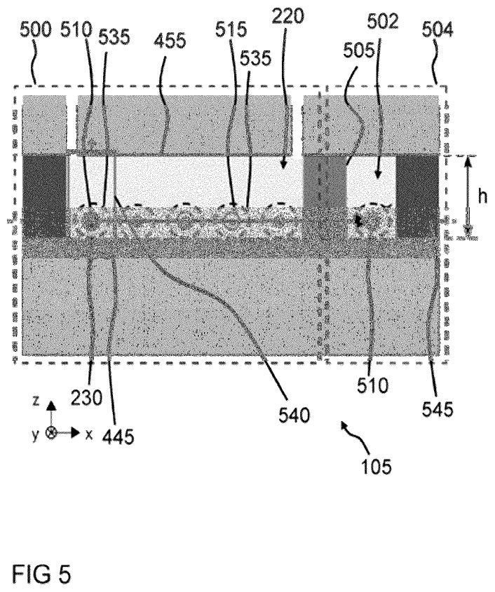

FIG. 5 a schematic cross-sectional view of a carrier substrate;

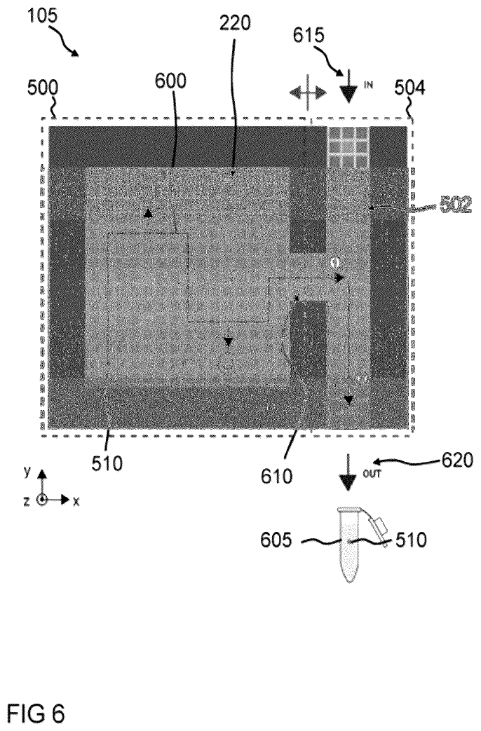

FIG. 6 a schematic illustration of a carrier substrate;

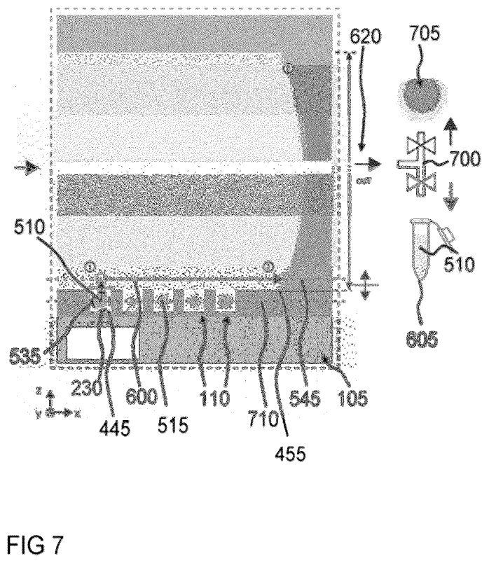

FIG. 7 a schematic cross-sectional view of an exemplary embodiment of a carrier substrate;

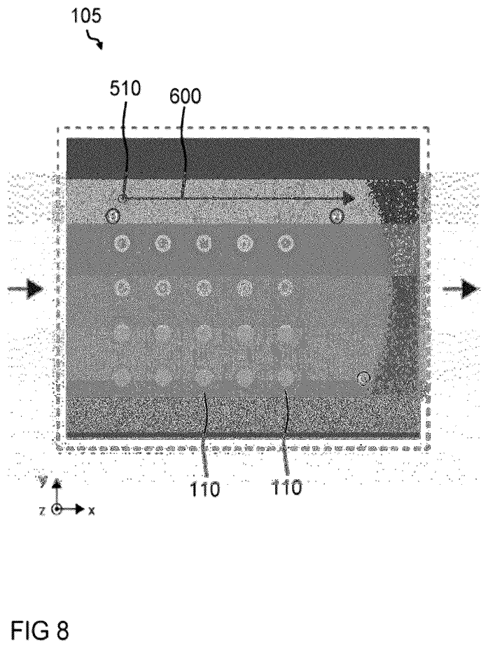

FIG. 8 a schematic illustration of an exemplary embodiment of a carrier substrate;

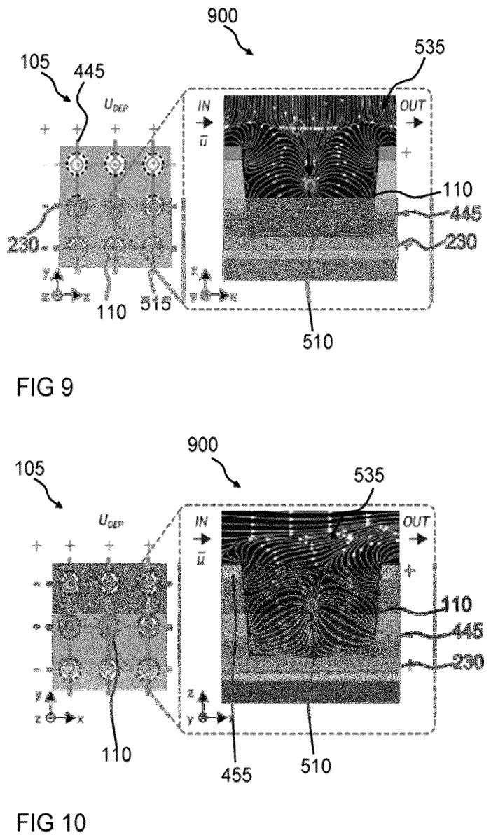

FIG. 9 a schematic illustration of an operating mode according to an exemplary embodiment for a microfluidic device;

FIG. 10 a schematic illustration of an exemplary embodiment of an operating mode for a microfluidic device;

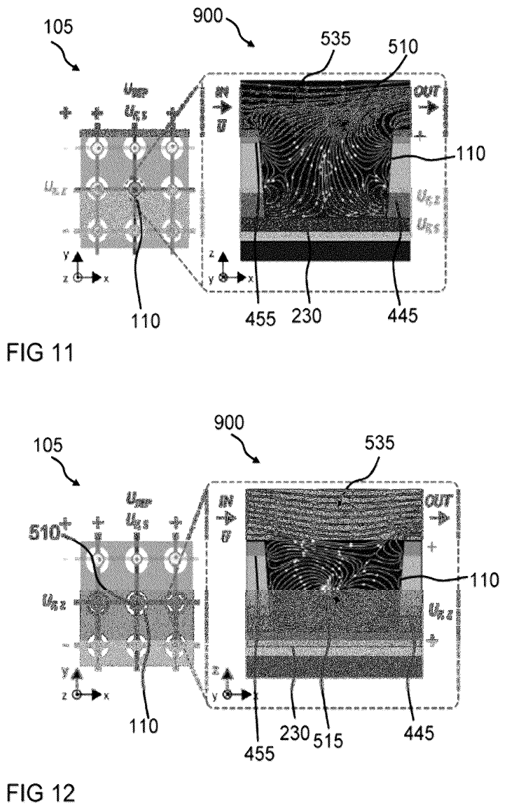

FIG. 11 a schematic illustration of an exemplary embodiment of an operating mode for a microfluidic device;

FIG. 12 a schematic illustration of an exemplary embodiment of an operating mode for a microfluidic device;

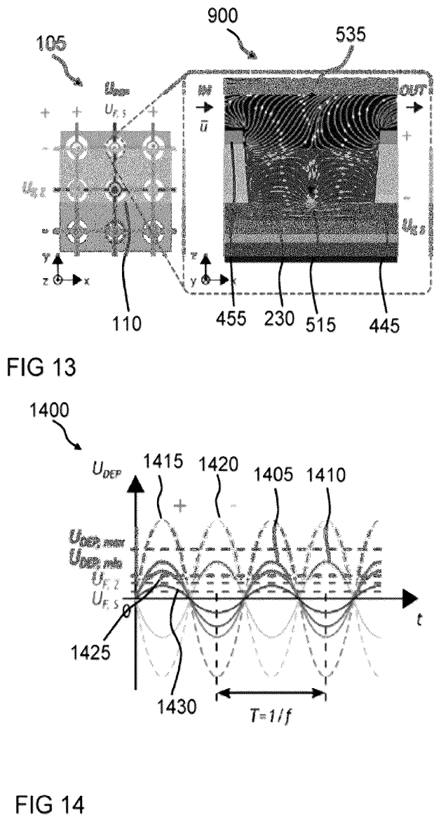

FIG. 13 a schematic illustration of an exemplary embodiment of an operating mode for a microfluidic device;

FIG. 14 a schematic diagram illustration of a voltage curve according to an exemplary embodiment for a microfluidic device;

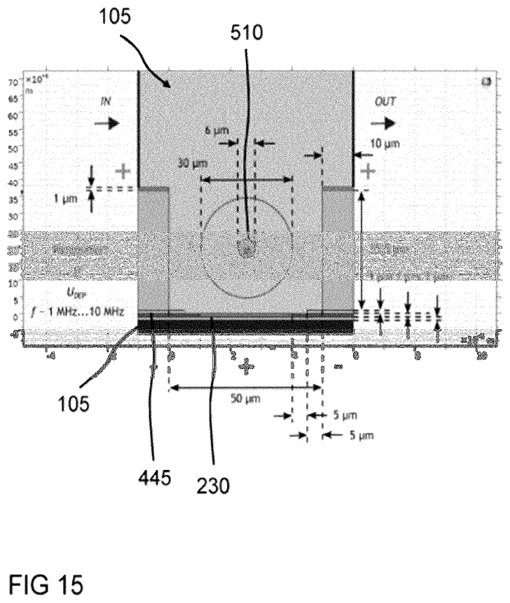

FIG. 15 a schematic illustration of a microcavity with dimensions according to an exemplary embodiment;

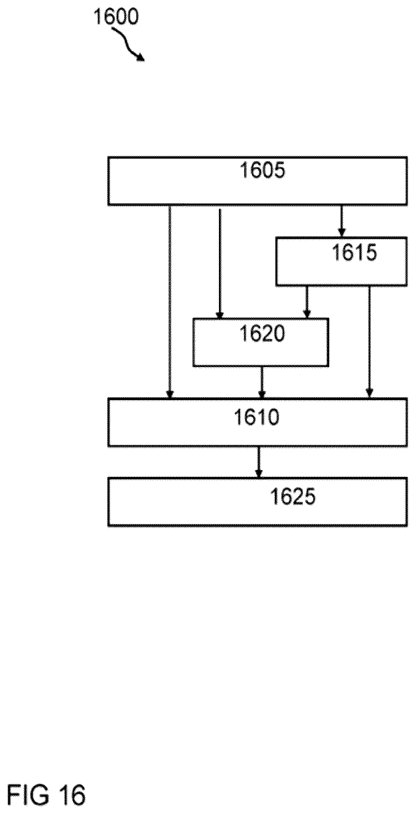

FIG. 16 a flowchart of a method according to an exemplary embodiment for trapping at least one nucleated cell using at least one electrode for a microfluidic device;

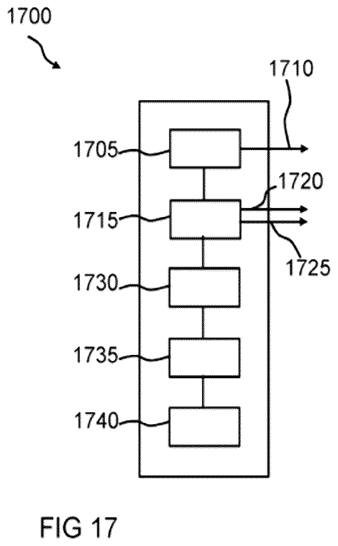

FIG. 17 a block diagram of a device according to an exemplary embodiment;

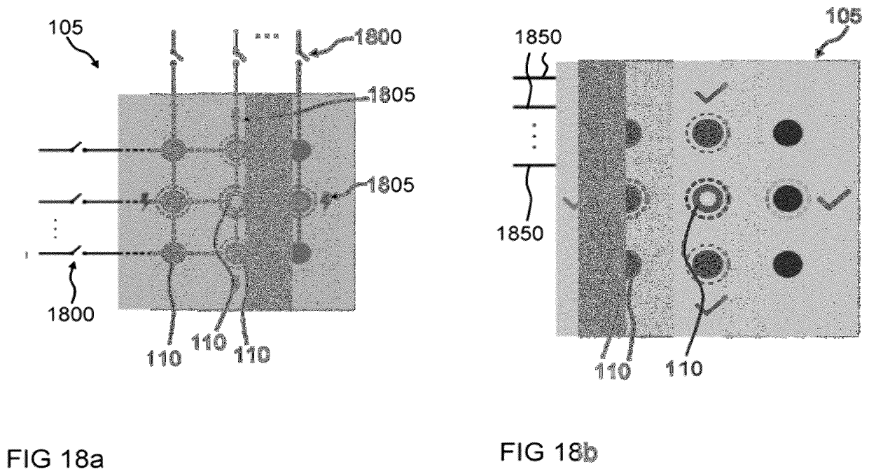

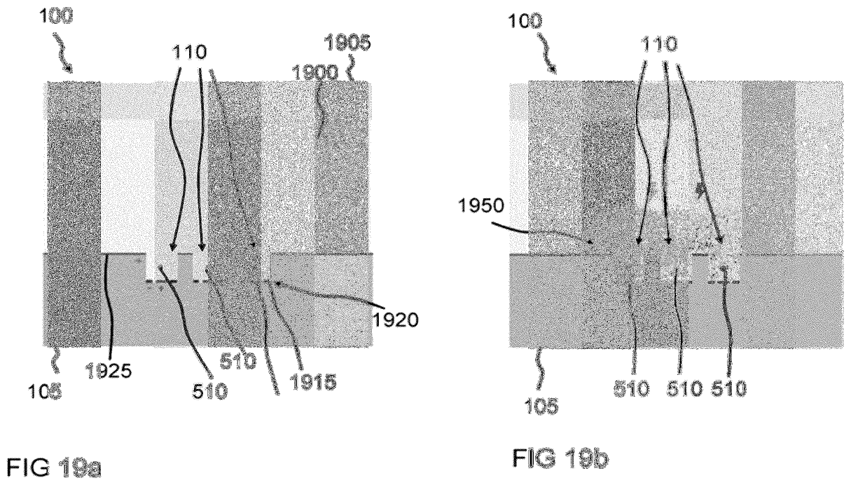

FIG. 18a a schematic illustration of an exemplary embodiment of a carrier substrate;

FIG. 18b a schematic illustration of an exemplary embodiment of a carrier substrate;

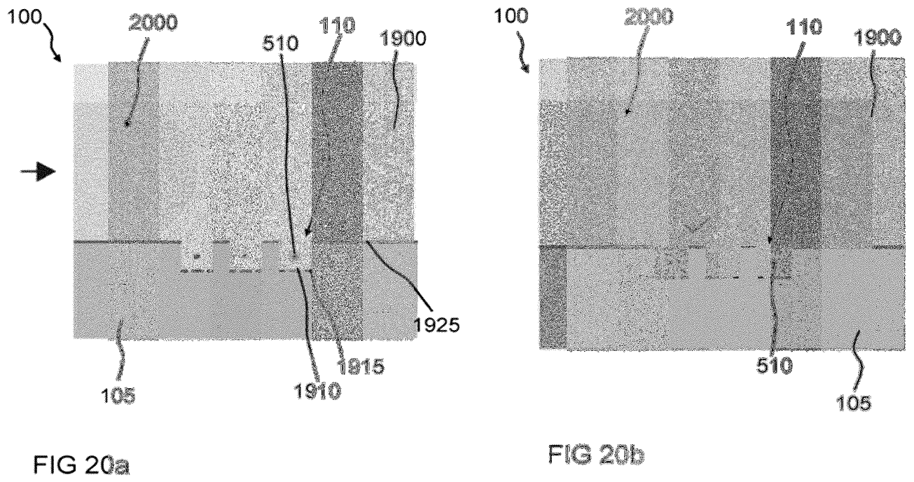

FIG. 19a a schematic cross-sectional view of an exemplary embodiment of a detection chamber of a microfluidic device;

FIG. 19b a schematic cross-sectional view of an exemplary embodiment of a detection chamber of a microfluidic device;

FIG. 20a a schematic cross-sectional view of an exemplary embodiment of a detection chamber of a microfluidic device; a

FIG. 20b a schematic cross-sectional view of an exemplary embodiment of a detection chamber of a microfluidic device.

In the following description of favorable exemplary embodiments of the present approach, identical or similar reference signs are used for the elements shown in the various drawings having similar functions, whereby a repeated description of these elements has been omitted.

FIG. 1 shows a schematic illustration of a microfluidic device 100 according to an exemplary embodiment of the invention. The microfluidic device 100 is in this case in particular designed as a lab-on-chip cartridge that is used, e.g., in conjunction with rapid tests to screen sample liquids for, e.g., tumor cells also referred to as circulating tumor cells (CTCs). The sample liquid is, e.g., blood from a patient. The microfluidic device 100 in this case comprises a carrier substrate 105 for holding the sample liquid. The carrier substrate 105 has at least one microcavity 110, preferably a plurality of microcavities 110. At least one electrode 115 is arranged in or on the microcavity 110 in order to generate an electric field. The at least one electrode 115 is in this case, e.g., point-like, ring-like, and/or layer-like in shape. The term “layer-like” means that the at least one electrode 115 is designed as, e.g., a single layer. The electric field is designed to trap a nucleated cell in the microcavity 110. According to this exemplary embodiment, the sample liquid is illuminated using an illumination unit 120, e.g., part of a microscope or an evaluation device.

According to this exemplary embodiment, the carrier substrate 105 is arranged on a printed circuit board 125, which in turn is arranged on a housing element 130 of the microfluidic device 100. According to this exemplary embodiment, the carrier substrate 105 has a width b and a length l with the same or similar values, so that it has a polygonal, in particular square, shape according to this exemplary embodiment.

In other words, the present approach describes an all-in-one system, the microfluidic device 100, which is designed to perform fully automated quantification and single cell dielectrophoretic sorting of nucleated cells, and in particular live circulating tumor cells, from whole blood using electrified microcavities 110.

A time-resolved and standardized quantification of CTCs enables following a patient's disease progression in real time, which is also referred to as real-time monitoring. As a result, it is possible to make therapy decisions (precision medicine) or even provide prognoses of progression-free survival that are adapted to the individual disease situation. Single cell sorting enables single cell analysis of such nucleated cells on a molecular and cellular plane. Furthermore, it is possible to isolate detected tumor cells from a contaminating background of healthy blood cells by suitable single cell sorting in a high quality, i.e., fast, efficient and remaining in a viable state, and to make them available for subsequent molecular genetic analyses, e.g. expression profiling, PCR, NGS, or e.g. subcultivation, stem cell and chemosensitivity tests. In such cases, the more precise information about the cancer is obtained. This concerns, e.g., the type and heterogeneity of the mutations that take place or possible resistance to the drugs used.

With this in mind, the approach presented here features microcavities 110 at the bottom of a relatively shallow and large area detection chamber. The resulting “all-in-one system” enables not only sample preprocessing and (basically) isolation-free quantification of living circulating tumor cells from healthy blood cells, but additionally dielectrophoretic single cell sorting of detected tumor cells for subsequent single cell analyses. The system is used in microfluidic environments and enables fully automated processing of whole blood.

Core elements of the approach presented in this context comprise sample preprocessing for quantification of CTCs and single cell sorting for single cell analysis of CTCs on the molecular and cellular planes. According to this exemplary embodiment, sample preprocessing and CTC quantification are performed in electrified microcavities 110 which can be used for single cell sorting. According to this exemplary embodiment, single cell sorting is based on non-contact manipulation by negative dielectrophoresis. For these purposes, individual cavities 110 are defined, e.g., by holes at intersections of capped traces arranged as electrically separated and exclusively addressable rows and columns within a matrix on the carrier substrate 105. The release of a single target cell or, depending on the sorting strategy, a non-target cell that has previously sedimented into a cavity 110 and is stably trapped there within a “DEP cage”, even during a rinsing operation, is accomplished by controlling the associated row and column using a suitable electric signal as a transfer of the cell from the trapping plane to the transport plane (use of a “DEP levitator”). Non-target cells or, depending on the sorting strategy, target cells in adjacent cavities 110 along the same row and column are not affected in their trapping states in the process. Released cells are transported away microfluidically, and due to a repulsive dielectric interaction, they do not come into contact with the chip for the entire duration of single cell sorting, nor are they flushed into adjacent DEP cages.

Since the DEP cages required for cell manipulation do not necessarily extend over the entire chamber height, but are formed within microcavities 110 at the bottom of the chamber according to one exemplary embodiment, decoupling between the electrical and microfluidic components is present. As a result, the chamber height and thus the specimen volume can be selected to be as large as desired, with otherwise constant footprint and supply voltages or DEP forces being achieved. Furthermore, since cells within microcavities 110, as compared to those above a planar substrate 105, experience (greatly) reduced Stokes forces as the medium in which they are suspended moves, with the presence of DEP cages within cavities 110 resulting in all the more stable trapping positions, efficient and lossless washing is quickly feasible. As a result, overall automated preprocessing of whole blood and quantification of circulating tumor cells by microfluidics is supported.

Since static DEP cages, i.e., cages whose position and size are fixed, can be used for single cell sorting, there is no need to use integrated active circuit components if they are suitably controlled. Such a passive chip can be manufactured comparatively easily and inexpensively. A purely fluidic removal of cells enables higher transport speeds than would be possible purely dielectrically. In addition, the transport speed is independent of the loading of the chip, since released cells no longer have to pass other cells in the same plane, but can simply fly over other cells in a straight line. Given these aspects, both positive selection and negative selection are equally practicable.

FIG. 2 shows a schematic diagram of a printed circuit board 125 according to an exemplary embodiment comprising a carrier substrate 105. The printed circuit board 125 shown in this case and/or the carrier substrate 105 arranged on the printed circuit board 125 are at least the same as or similar to the printed circuit board 125 described in FIG. 1 and/or the carrier substrate 105 described in that case. The printed circuit board 125 is in this context also referred to as a PCB carrier, e.g., having a carrier region 200 and an interposer region 205. According to this exemplary embodiment, the carrier substrate 105 is designed as a silicon chip that is electrically connected, e.g. with the interposer region 205, to an external control circuit, e.g. contacted or contactable via a plurality of connection interfaces 210, which is also referred to as bonding. The connection interfaces 210 are in this case designed to, e.g., electrically contact the electrode(s) arranged on or in the carrier substrate 105. The control circuit is also hereinafter referred to as the device. The interposer region 205 has at least one adjustment hole 215, in particular two, designed to suitably position and fix, e.g. press or screw on, an interposer.

For example, a Si chip bonded to a carrier PCB, referred to herein as PCB 125, can be microfluidically integrated into a LoC cartridge and electrically contacted to an external control circuit via an interposer system.

For example, the chamber height required for a chamber with a footprint of 12.5 mm×12.5 mm is at least 320 μm to accommodate blood lysate with a total volume of 50 μL. However, if required, higher chambers can also be achieved without any problems, which then lead to correspondingly larger sample volumes. For example, for a chamber of height >1 mm and footprint of ˜12.5 mm×12.5 mm, a chamber volume of >156 μL is obtained.

According to this exemplary embodiment, the carrier element 105 has a plurality of layers 220, and a structure of the carrier substrate 105 is described in more detail in one of the following drawings. According to this exemplary embodiment, the carrier substrate 105 has a chamber 225 in which the sample liquid is analyzed. According to this exemplary embodiment, the microcavities 110 are arranged in the region of the chamber 225. Furthermore, each of the microcavities 110 has at least one electrode 230 which can, e.g., be individually controlled. Optionally, the microcavities 110 are arranged in a matrix-like manner on the carrier substrate 105.

FIG. 3 shows a schematic diagram of an interposer region 205 according to an exemplary embodiment. The interposer region 205 shown in this case is the same or at least similar to the interposer region 205 described in FIG. 2. According to this exemplary embodiment, the interposer region 205 is thereby shown in an exploded view 300 and a cross-sectional view 302.

The printed circuit board 125 is thereby designed as a base element on which the interposer 303 (which comprises, e.g., an intermediate element 305 and/or a cover element 310) is arranged. According to this exemplary embodiment, the interposer 303, the intermediate element 305 and the cover element 310, and the printed circuit board 125 are pressed together.

According to this exemplary embodiment, a modular structure is shown: An electrically passive sorting chip, which in this case consists of a printed circuit board 125 and a carrier substrate not visible in FIG. 3, is contacted with an active control circuit by pressing it against the interposer 303 for the duration of the single cell sorting. Each ground electrode and the counter-electrode on the passive chip are assigned a contact pad that can be controlled exclusively. For example, to achieve 10000 DEP cages in a classical matrix, 100+100+1=201 contact pads are necessary. An exemplary size of a contact pad is 500 μm×500 μm. For electrified microcavities implemented in PCB technology, the necessary contact pads are provided directly on the same PCB 125. If, on the other hand, Si technology is used, an additional carrier PCB is recommended, on which the necessary contact pads are applied. This means an electrical connection between Si chip and carrier PCB is made, e.g. via bonding wires.

While the passive part is located, e.g., within a lab-on-a-chip microfluidic system described herein as a microfluidic device, the active part is only optionally located within an associated and spatially separated evaluation unit.

FIG. 4 shows a schematic assembly diagram of an exemplary embodiment of a carrier substrate 105. In this context, the carrier substrate 105 shown in this case is the same or similar to the carrier substrate 105 described in one of FIG. 1 or 2. According to this exemplary embodiment, the carrier substrate 105 has a plurality of layers 220 shown in six Subfigures a) to f). According to this exemplary embodiment, Subfigures a) to f) are understood as building upon one another.

Subfigure a) in this case shows a base material 400 of the carrier substrate 105, e.g. silicon, which comprises a first oxide layer 405 and thus serves as a base. Subfigure b) in this case corresponds to Subfigure a). Merely in deviation, the carrier substrate 105 in Subfigure b) according to this exemplary embodiment additionally has a metal layer 410 comprising a plurality of metal rods 415 or, e.g., metal strips. The metal layer 410 is in this case arranged on the oxide layer 405 such that it acts as the at least one electrode for each of the microcavities to be formed. According to this exemplary embodiment, the metal rods 415 are spaced apart so that a gap S is arranged between each of them.

Subfigure c) shows the carrier substrate 105, which according to this exemplary embodiment corresponds to the carrier substrate 105 shown in Subfigure b). Merely in deviation therefrom, the carrier substrate 105 according to this exemplary embodiment additionally comprises a second oxide layer 420 having a plurality of openings 412 along the metal rods 415 of the metal layer 410. These openings 412 are in this case arranged on the metal rods 415 such that they act as at least one electrode at cavity bottoms of the microcavities when the carrier substrate 105 is in an operational state. A diameter d of the openings 412 corresponds in each case to a width w of a metal rod 415.

According to this exemplary embodiment, Subfigure d) shows a further development of Subfigure c). In addition thereto, the carrier substrate 105 comprises another metal layer 425 that is also formed from a plurality of additional metal rods 430. The further metal rods 430 thereby extend transversely to the metal rods 415. In the region of the openings 412 of the second oxide layer 420, the further metal rods 430 comprise ring-shaped sections whose size and position depend on the size and position of the openings 412 and which, according to this exemplary embodiment, form at least one further electrode 445. According to this exemplary embodiment, the electrode 230 and the further electrode 445 are electrically insulated from each other and further optionally arranged concentrically. Additionally or alternatively, the electrodes 230, 445 are layer-like relative to each other. In other words, the at least one electrode 230 is arranged on the cavity floor and/or in a cavity wall of at least one of the microcavities 110.

Subfigure e) corresponds to Subfigure d) except that, according to this exemplary embodiment, a photoresist layer 450 is additionally arranged over the second metal layer 425. The photoresist layer 450, like the second oxide layer 420 before it, has further openings arranged at the position of the electrodes 230, 445. However, the further metal rods 430 are covered by the photoresist layer 450, which has, e.g., electrically isolating properties. According to this exemplary embodiment, the electrodes 230, 445 are spaced apart. According to this exemplary embodiment, a resulting bar has the same width w as each of the metal rods 415.

For example, the carrier substrate 105 shown in Subfigure f) corresponds to the carrier substrate 105 described in Subfigure e). Only differently, the carrier substrate 105 in Subfigure f) additionally has a counter-electrode 455 (which is in this case designed as a metal layer), which is arranged above the photoresist layer 450 and is thus realized as a third electrode.

In other words, a layer structure of the novel DEP chip in silicon technology with exemplary dimensions is shown. The chip is composed of a plurality of layers.

According to this exemplary embodiment, the microcavities 110 are achieved in thin film technology. In this case, the carrier substrate 105 is implemented, e.g., as a thermally oxidized silicon wafer. The ground electrodes 230, 445, which are formed by the metal rods 415, 430, and an oxide layer 420 electrically isolating them can be imaged lithographically, for example: Optionally, this can be done either in combination with a sputtering or waxing process and etching, or alternatively with a vapor deposition and lift-off. Horizontal resolutions of ˜1 μm are thereby easily achieved. The layer thicknesses for metal films and oxides, e.g., range from a few nanometers to a maximum of ˜3 μm. A counter-electrode 455, described herein as a metal layer, can be fabricated in the same manner and has the same fabrication limitations as the ground electrodes 230, 445 as well. The walls of cavities, e.g., are generated by a thick lithographically patterned photoresist, referred to here as photoresist layer 450 with dimensions from 2 μm up to 200 μm in depth with an aspect ratio of up to ˜1:10. The shape of the footprint of a cavity 110 can be as desired, e.g., circular, hexagonal or square. Cavities 110 can be arranged on the ground electrodes 230, 445 within a “classical matrix” or, alternatively, densely packed.

Alternatively, the microcavities 110 can be implemented in printed circuit board (PCB) technology as follows. For example, the carrier substrate 105 comprises a rigid FR4 carrier, e.g. >1 mm thick base material 400. The functional layers of the carrier substrate 105 are, e.g., bonded together via an adhesive. The metal layers or conductor tracks comprise, e.g., thin copper foils structured by photolithography and wet-chemically etched, which comprise, e.g., a thickness from 6 μm to 70 μm. Conductor track widths and spacings can, e.g., be achieved according to the standard starting from as little as 15 μm. Isolators between conductive tracks usually feature, e.g., thin polyimide films with thicknesses starting at 12 μm. Different metal layers and isolators are joined together via lamination processes. Drilled holes at crossing points of the ground electrodes 230, 445, also called “unplated blind vias”, are created, e.g., by ablation with an (ultra) short pulse laser. These have diameters starting at, e.g., about 15 μm and an aspect ratio of ˜1:1. Exposed metal surfaces, especially those in contact with the medium, can be protected by what is referred to as a “finishing” process. Standard end surfaces in PCB technology are e.g., ENIG, ENEPIG, EPIG or electroplated gold. It should also be noted at this point that the end surface in this exemplary embodiment is provided on all free-standing metal surfaces, including all electrodes 230, 445 and 455. It should be noted that finishing need not lead to electrical short circuits between different contacts. The microcavities 110 have, e.g., a diameter of 100 μm and a depth of 66 μm.

FIG. 5 shows a schematic cross-sectional view of a carrier substrate 105. The carrier substrate 105 shown in this case is the same as or at least similar to the carrier substrate 105 described in one of FIG. 1, 2, or 4. According to this exemplary embodiment, at least one chamber 220 is arranged in or on the carrier substrate 105 in a main section 500. Additionally, the carrier substrate 105 comprises a secondary chamber 502 in a secondary section 504 that is smaller than the chamber 220. The two chambers 220, 502 in this case have the same height h. The chamber 220 is separated from the secondary chamber 502 by a partition 505. The chamber 220 is thereby designed to receive the sample liquid. The sample liquid has at least one nucleated cell 510, but in particular also at least one further nucleated cell 515. The nucleated cell 510 is in the form of a tumor cell, and the further nucleated cell 515 is in the form of a leukocyte. The electrodes 230, 445 are further concentrically arranged in a bottom region of chamber 220. The carrier substrate 105 comprises the electrically conductive metal layer 455 designed as, e.g., a third electrode and arranged opposite the electrodes 230, 445. The electrodes 230, 445, and 455 are designed to generate an electric field 535 in a cage region 540 that acts on the nucleated cell 510 like a cage. As a result, the position of the nucleated cell 510 is fixed. Further, an electric field 535 acts on each of the nucleated cells 510, 515. It is therefore possible to transport the nucleated cell 510 past the at least one other nucleated cell 515 without them touching, thereby achieving single cell sorting. The nucleated cell 510 is thereby transported to the secondary chamber 502.

Traditionally, in order to make use of the information content of circulating tumor cells from blood samples, CTC quantification with the necessary sample preprocessing and, if subsequently pursued, CTC single cell analysis with the necessary single cell sorting are processes that are decoupled from each other. Doing so usually requires multiple apparatuses, e.g. independent platforms interconnected as “process stages,” and manual process steps using laboratory equipment, e.g. pipettes, reaction vessels, or centrifuges.

Sample preprocessing typically provides for, at a minimum, removal of red blood cells (RBCs) and fluorescent staining (labeling) of nucleated cells for optical detection and classification of CTCs. Depending on whether (further) sorting of CTCs from leukocytes (White Blood Cells, WBCs) is performed prior to CTC counting or not, is referred to as isolation-related or (basically) isolation-free methods.

Solutions for single cell sorting generally make a tradeoff between sorting throughput and sensitivity. The strategy chosen depends largely on the specific application, and it can involve the nature of the sample in its initial state, as well as the requirements of subsequent single cell analysis.

Regarding the sorting of larger cell quantities with, e.g., a starting quantity of, e.g., more than one million cells, fluorescence-based flow cytometers (FACS) are traditionally used. However, these have comparatively large dead volumes and can exhibit relatively large losses, depending on the size and intensity of the markers being detected on the cells. Furthermore, the option of visual control (imaging) is normally not available during the sorting process. Apart from microfluidic systems, individual target cells detected and possibly enriched in a precursor, e.g. circulating tumor cells, can also be isolated from non-target cells, e.g. healthy blood cells like leukocytes, classically for each microcapillary (“cell picker”). For this purpose, ultrafine capillaries usually made of glass with an inner diameter in the range of a few 10 μm, e.g., are moved to the target cell with the aid of a micromanipulator. By applying a well-defined negative pressure, the target cell is aspirated from the surrounding non-target cells and transferred, e.g., to a separate vessel for further analysis.

If one is interested in the efficient sorting of small biopsies with, e.g., a starting quantity of a few tens of thousands of cells, which in turn corresponds, by way of example, to the number of leukocytes in a few microliters of blood, in a microfluidic environment, the approach of manipulating biological cells on the basis of their dielectric properties has so far emerged as promising. The underlying mechanism, known as dielectrophoresis (DEP), refers to the movement of even uncharged polarizable particles in a spatially non-homogeneous electric DC or AC field. The externally applied electric field in this case interacts with electric multipoles induced by this same external field, and a dielectrophoretic force effect is produced on the particle.

Biological cells are manipulated in this way without contact and independently of markers. Furthermore, since the effect is scalable on a large scale, it results in good compatibility with modern MEMS technologies. In the simplest case of a spherical particle—exemplarily representative of a living circulating tumor cell to be isolated from the cellular blood components—the

$${\tilde{f}}_{CM} = \frac{{\tilde{ε}}_{p} - {\tilde{ε}}_{m}}{{\tilde{ε}}_{p} + 2 {\tilde{ε}}_{m}}$$

time-averaged 1st order dielectrophoretic force (the term is sufficient for moderately inhomogeneous electric fields in describing dielectrophoresis) can be expressed in the most general case for a spatially stationary electric field as:

<\vec{F}_{DEP}>=2πεₘ·Re(\tilde{f}_{CM})·R³·\vec{∇}|\vec{E}_{RMS}|²

R stands in this case for the radius of the particle under consideration, and \tilde{ε}p and \tilde{ε}m stand for the absolute complex electrical permittivities of particle p and the surrounding medium m, where

$$\tilde{ε} = ε - j \frac{σ}{ω} ,$$

ε represents the absolute real electric permittivity j=\sqrt{−1} as a complex unit, σ is the electric conductivity and ω is the angular frequency of the applied electric field. Depending on the sign of the (real part of the) Clausius-Mossotti factor Re(\tilde{f}_{CM})—this depends on the frequency of the electric field and the relative tuning between frequency-dependent absolute real electric permittivity ε and electric conductivity σ between medium and particle (−interior)—for manipulation either an attractive (positive dielectrophoresis resp. pDEP with effect in direction of maxima of electric field strength, Re(\tilde{f}_{CM}) maximum+1) or repulsive (negative dielectrophoresis or nDEP with effect in direction of minima of electric field strength, Re(\tilde{f}_{CM}) minimum −½) force effect on individual particles can be induced for manipulation.

If the approach of manipulating cells via pDEP is pursued, then it should be kept in mind that this is only achievable in a low conductivity buffer. Although on the one hand this leads to (significantly) lower heat losses overall, on the other hand this measure is associated with an additional, separate deionization process and the enforced “artificial atmosphere” can have a negative effect on the viability and proliferation behavior of cells (“leakage of the cell interior”).

Stable force equilibria always arise for attractive DEP forces sterically at the field-generating electrode surfaces or, more generally formulated, at locations of maximum electric field strength on a chip surface. DEP traps are much easier to generate and stronger in this way, in particular the restraint force for increasing voltages increases, but this leads to contact and thus possibly adhesion of cells with the chip surface and cells can be lysed by excessive electric field strengths. These are sometimes the decisive disadvantages that can cause a (strongly) reduced efficiency or purity of sorting by pDEP. In striving to circumvent the problems just listed, negative DEP forces can alternatively be used to sort cells, as is “only” achieved in physiological media, e.g., blood plasma, PBS, or cell culture medium. Although an increased conversion of electrical power into heat must then be expected, which can only be dissipated by suitable additional components, e.g. fans or Peltier elements, there is no need for prior deionization. Furthermore, maintaining a natural medium maximally conserves cell viability and expression profiles.

In the case of repulsive DEP forces, cells are always moved to locations of minimum field strength, usually away from the substrate. This prevents contact between cells and the chip surface and counteracts sticking and the destruction of cells by field overshoots. In this context, the achievement of a stable force equilibrium within a sufficiently strong DEP trap, whose restraining force usually decreases with increasing tension, turns out to be quite challenging. The strategy used in the present approach is to isolate target cells from non-target cells in one plane, but using two separate chambers. A sorting process comprises the following two steps:

First, the prepared cell suspension of interest is introduced into a large main chamber, also referred to herein as chamber 220 and further referred to as “chamber 1”, and the adjacent small secondary chamber 502, further referred to as “chamber 2”, is filled with a clean wash buffer of physiological composition. For the filling operations, the individual chambers 220, 502 are provided with an inlet for input and an outlet for deaeration or output. Both chambers 220, 502 are fluidically connected to each other without air after filling. The cells 510, 515 in the chamber 1 are randomly but homogeneously distributed; no cells enter chamber 2. In chamber 1, target cells are trapped purely (di-) electrically and transported “deterministically”, i.e., along individually programmable trajectories, past equally trapped non-target cells in a fixed plane 545 without collision until they reach chamber 2. For example, chambers 220, 502 and their walls are defined by a double-sided adhesive tape of suitable height and layout, which connects the floor and the ceiling in a sealing manner. The transport of target cells takes place contactlessly in and using three-dimensional “permanently closed DEP cages”. Such a cage in the “standard configuration” consists of, e.g., 3×3 planar square silicon electrodes 230, 445, located on the chamber floor (each electrode: ˜18.8 μm×18.8 μm with 1.2 μm spacing to adjacent electrodes), and a transparent indium tin oxide (ITO) counter-electrode 455 extending over the entire surface of the chamber ceiling. To generate a field minimum in the middle of the chamber into which cells are stably placed via nDEP, the central ground electrode 230 and the counter-electrode 455 on the chamber ceiling are set to an equal alternating potential (“in-phase” or “+”), while the outer ground electrodes 445 are operated in opposite phase to this (“counter-phase” or “−”).

In chamber 2, target cells 510 previously “ejected” from chamber 1 are dispensed into external reaction vessels, e.g. Eppendorf Tubes, via a final rinse with a clean buffer. This process is then performed purely microfluidically and therefore “non-deterministically”, i.e., the nucleated cells merely follow a rectilinear trajectory according to this variant.

The sorting process presented in this case ensures reproducible efficiencies and purities of 100% in each case, but carries with it “consequences”, which should be interpreted as follows:

In order to generate a sufficiently high electric field strength or DEP force for cell manipulation within the framework of easily achievable and manageable supply voltages (U_{DEP}≤5 V_{RMS}) that do not yet induce electrolysis effects, the height of the chamber 1, and in connection with this the maximum sample volume that the chamber 1 can hold, is limited. For example, a maximum ˜100 μm chamber height for a chamber 1 footprint of ˜12.5 mm×12.5 mm results in a maximum ˜15.6 μL chamber volume. Furthermore, unlike the case in chamber 2, efficient washing in chamber 1 is not guaranteed or practical. For these reasons, external concentration with additional enrichment of target cells within a clean buffer is provided. This is usually accomplished by a centrifuge, thus requiring manual sample transfer between apparatuses and incurring additional cell losses. The “dynamic” DEP cages required for actuation, i.e., those whose position and size can be varied, as described hereinabove, can only be achieved by active components, e.g. transistors or memory elements. These are integrated within the individual silicon electrodes in CMOS technology.

According to this variant, a maximum transport speed for target cells in chamber 1 is limited (˜1 electrode width/second) because the (lateral) DEP forces used for transport are limited. Furthermore, as an option, the maximum transport speed depends on the chip load in each individual case. In other words, although, compared to the standard configuration, a closed DEP cage for accommodating theoretically a total of

$$\frac{3 \times 3}{2 \times 2} = \frac{9}{4} = 2.25$$

times more cells can also be generated using only a pattern of 2×2 electrodes (“reconfiguration”), target cells in this case first require “clearing a path to the exit”, which is associated with an increase in sorting time. Negative selection is neither intended nor practical.

FIG. 6 shows a schematic diagram of a carrier substrate 105. The carrier substrate 105 shown in this case corresponds to, e.g., the carrier substrate 105 described in FIG. 5. Only their perspective of presentation differs. In other words, the carrier substrate 105 is shown from the top view. By way of example only, a trajectory 600 is shown along which the nucleated cell 510 is transported, using electric fields and changing voltage values, first into the secondary section 504 and then out of the carrier substrate 105 into, e.g., a sample vessel 605.

The main section 500 and the chamber 220 are in this case square in shape. Further, the chamber 220 is optionally connected to the secondary chamber 502 via a bottle neck type connecting section 610. The secondary chamber 502 is in this case substantially rectilinear in shape and comprises an inlet 615 and an outlet 620 arranged opposite the inlet 615. For this purpose, there is a separate inlet-outlet pair (IN 615 and OUT 620), whereby this inlet-outlet pair is only intended for rinsing/transport/outlet operation with a clear buffer. The outlet 620 is designed to discharge the singulated nucleated cell 510 from the carrier substrate 105 into the sample vessel 605 via the secondary chamber 502.

FIG. 7 shows a schematic cross-sectional view of an exemplary embodiment of a carrier substrate 105. The carrier substrate 105 shown in this case is, e.g., applicable to a microfluidic device as described by way of example in FIG. 1. The carrier substrate 105 is also at least similar to the carrier substrate 105 described in one of FIGS. 1, 2, 4, 5, 6. According to this exemplary embodiment, microcavities 110 are shown arranged as depressions in the carrier substrate 105. According to this exemplary embodiment, the carrier substrate 105 shown in this case also has concentrically arranged electrodes 230, 445 formed to form the electric field 535. In order to be able to form the electric field 535 (“DEP cage”) in a very advantageous manner, the flat electrode can also be used as a counter-electrode 455, which in this case forms a third electrode on the roof of the microcavity 110 and is arranged opposite the electrode 230. According to this exemplary embodiment, all of the microcavities 110 are constructed in the same way, so electric fields can be generated in each of them. Doing so enables, e.g., single cell sorting on the carrier substrate 105. Doing so further enables the nucleated cell 510 to be transported out of the microfluidic device along a trajectory 600. According to this exemplary embodiment, the outlet 620 is connected to a valve 700 designed as, e.g., a double valve. The valve 700 is designed to direct the nucleated cell 510 into the sample vessel 605 and to drain an unwanted liquid 705, e.g. lysate residue, from the sample liquid.

In other words, single cell sorting using electrified microcavities 110 is described, whereby nucleated cell 510 are isolated from non-target cells, i.e., from further nucleated cells 515 in a chamber, but using two separate planes 545, 710. A corresponding sorting process provides in this case for a preprocess and spatial separation, thus enabling quantification of the nucleated cell 510.

According to this exemplary embodiment, the carrier substrate 105 comprises electrified microcavities 110 for single cell sorting. Outside, a 2-way valve, also referred to in this case as valve 700, is provided. The valve 700 is designed to completely spatially separate the blood lysate 705, which is to be washed away in the preprocess and acts as a contaminant, from the nucleated cell 510 using the downstream clean wash buffer during single cell sorting. Alternatively, it is conceivable to use two different reaction vessels to separate the blood lysate 705 without valve 700, which are placed sequentially at the outlet, that is, at outlet 620.

At the bottom of a single microcavity 110 is a first point-shaped electrode 230, which is electrically insulated and surrounded by a second annular electrode 445. These two ground electrodes 230, 445 are concentric and coincide with the center of gravity of the footprint of the microcavity 110. Electrodes 230, which are point-like electrodes 230 passing through cavities 110, form the gaps. For this purpose, electrodes 445, which are twisted by 90° and extend through the cavities as ring-shaped electrodes, form the rows of a matrix in which each ground electrode 230, 445 can be controlled separately. Finally, in order to be able to generate a three-dimensional “DEP cage with release function”, a third full-surface counter-electrode, also referred to as metal layer 455, is required to form a bar top between adjacent tubs.

According to this exemplary embodiment, near-bottom and randomly, but uniformly, distributed intact nucleated cells 510, 515, meaning leukocytes and circulating tumor cells, are guided into stable floating positions in the middle of the microcavities 110 in the course of sedimentation without contact when the medium is at rest (ū=0), if all point-shaped ground electrodes 230 and the counter-electrode 455 are set to a sufficiently high and equal alternating potential, while all ring-shaped ground electrodes 445 are operated in equal and opposite phase for this purpose. However, the voltage levels U_{DEP,min} must be selected low enough at the same time to obtain open DEP cages.

Immediately before a wash (ū>ūₘₐₓ), the voltage is increased to a given maximum value U_{DEP,max} with otherwise unchanged signal assignment at the ground electrodes 230, 445—closed DEP cages with maximum holding force are created, in which all cells 510, 515 remain stably trapped against the prevailing flow forces without coming into contact with the walls or the bottoms of the cavities 110. As a result, intact cells located outside a cage can no longer enter it, while all cells 510, 515 remain stably trapped inside a cage. The washing process results in a chamber completely free of contamination and is only optional.

Optionally, and without being generally limiting, the process of positive selection, i.e., isolation of a target cell 510 from non-target cells 515, is presented hereinafter. Conversely, of course, negative selection is achieved in a similar manner by releasing non-target cells 515 from microcavities 110 while target cells 510 are not.

All of the cells 510, 515 sedimented to the bottom of the chamber are initially in an equilibrium position within the microcavities 110 in the initial state, i.e., at a constant height starting from the bottoms of the cavities 110. This lower trapping plane will be referred to as plane 710 for further consideration. The level of the applied DEP voltage is still maximum, the medium inside the chamber is still in motion. To eject a target cell 510, 515 from a stable trapping state in plane 710 and transfer it to an upper transport plane (denoted in this case as plane 545) above a bar top, the ground electrodes 230, 445 crossed at the location of the target cell 510 are driven with suitable electric signals. For this purpose, a release voltage U_{F,S} is applied to the associated column and a release voltage U_{F,Z} is applied to the associated row for this purpose. The release stresses are geometry-dependent. When 0≤U_{F,S}≤UF,Z≤U_{DEP,max}, the release voltages are chosen as follows to ensure a purely (di-) electrical release from a single cell from its floating position “deterministically”, but without affecting neighboring cells in their trapping positions. All signals used relate to, e.g., constant frequency harmonic excitation.

Cell release requires net a sufficiently large repulsive DEP force in the negative z-direction along the central axis of symmetry through microcavity 110 (“DEP levitator”). For this purpose, for a given geometry of the microcavity 110, a suitable combination of U_{F,S} and U_{F,Z} ensures that the lines of electric field strength from the counter-electrode 455 all fall on the exposed surface of the point-shaped ground electrode 230 (column) as parallel as possible to a z-axis and that there is still a sufficiently large field strength. Disturbances of this field curve by the annular ground electrode 445 (row) in the edge region of the microcavity 110 near a wall must be suppressed as far as possible.

U_{F,Z} should at the same time be chosen small enough to avoid DEP cage collapse of non-target cells 515 along the release line, i.e., to the left and right of cavity 110 of target cell 510, and to maintain closed DEP cages with sufficient holding force. U_{F,S} on the other hand, is selected to be large enough that, in a temporary new equilibrium state within lowered closed DEP cages, there is no contact between non-target cells 515 and the bottoms of the associated cavities 110 along the release gaps, i.e., above and below the cavity 110 of the target cell 510.

If released cells previously found protection in plane 710 within cavities 110 and experienced only reduced overall Stokes forces during washing, the situation changes in plane 545. In this case, the (downstream) transport to the outlet 620 and the output with a clean (wash) buffer is purely microfluidic, i.e. non-deterministic, by maximum Stokes forces and thus relatively fast. Released cells cannot enter neighboring DEP cages because they are closed. The sorting principle thus enables maximum efficiencies and purities in total.

FIG. 8 shows a schematic diagram of an exemplary embodiment of a carrier substrate 105. The carrier substrate 105 shown in this case is, e.g., the same or similar to the carrier substrate 105 described in one of FIG. 1, 2, or 4 to 7. The only difference according to this exemplary embodiment is the illustrated perspective. In other words, the carrier substrate 105 shown in this case is shown from a top view. Again, the carrier substrate 105 comprises a plurality of microcavities 110. In this case, too, the microcavities 110 are arranged in a matrix-like manner, i.e., in rows and columns. Again, a trajectory 600 for the at least one nucleated cell 510 is shown.

FIG. 9 shows a schematic diagram of an exemplary embodiment of an operating mode 900 for a microfluidic device. At least the carrier substrate 105 shown in this case is similar to the carrier substrate 105 described in one of FIG. 1, 2, or 4 to 8. According to this exemplary embodiment, the operating mode 900 refers to a state in which the electrodes of the microfluidic device are activated and form the electric field 535. According to this exemplary embodiment, the operating mode 900 is illustrated using a cross-sectional view of a microcavity 110 of the carrier substrate 105 with electrodes 230, 445, and 455 arranged in layers. The electrodes 230, 445, and 455 are in this case arranged at or within each individual microcavity 110. They are in this case thereby arranged such that, in the activated state of the electric field 535, the nucleated cell 510 enters a center of the microcavity 110. In other words, an electrical cage is open.

In other words, according to this exemplary embodiment, an operating mode 900 of single cell sorting using PCB electrified microcavities 110 is illustrated and described, e.g. positive selection, which describes isolation of a target cell 510 from non-target cells 515.

FIG. 10 shows a schematic diagram of an exemplary embodiment of an operating mode 900 for a microfluidic device. The microcavity 110 shown in this case is, e.g., similar to the microcavity 110 described in FIG. 9. Only the electric field 535 is shown differently according to this exemplary embodiment. According to this exemplary embodiment, the electrical cage is shown closed, so the nucleated cell 510 is trapped in its position centered in the microcavity 110. For example, a liquid present around the nucleated cell 510 can be rinsed out without washing the nucleated cell 510 out of the microcavity 110 (=washing process). The same is true of the nucleated non-target cells 515.

FIG. 11 shows a schematic diagram of an exemplary embodiment of an operating mode 900 for a microfluidic device. The microcavity 110 shown in this case is, e.g., similar to the microcavity 110 described in FIG. 10. Only the electric field 535 is shown differently according to this exemplary embodiment. According to this exemplary embodiment, the electrodes 230, 445, and 455 are controlled such that the nucleated cell 510, also referred to as the target cell, can be electrically pushed out of the microcavity 110 and then microfluidically flushed out already above the microcavity by flushing. This is achieved by, e.g., changing an electrical voltage U applied to the electrodes.

FIG. 12 shows a schematic diagram of an exemplary embodiment of an operating mode 900 for a microfluidic device. The microcavity 110 shown in this case is, e.g., similar to the microcavity 110 described in FIG. 11. Only the electric field 535 is shown differently according to this exemplary embodiment. According to this exemplary embodiment, electrodes 230, 445, and 455 are driven such that another nucleated cell 515, also referred to as a non-target cell, is held in the microcavity 110. Changing the voltage releases, e.g., the further nucleated cell 515, or alternatively holds it in place in the microcavity 110. This is achieved by, e.g., changing an electrical voltage U applied to electrodes 230, 445, and 455. The voltage U required for this is optionally different from the voltage U required for the nucleated cell 510. As a result, it is possible to, e.g., transport the nucleated cell 510 past the further nucleated cell 515.

FIG. 13 shows a schematic diagram of an exemplary embodiment of an operating mode 900 for a microfluidic device. The microcavity 110 shown in this case is, e.g., similar to the microcavity 110 described in FIG. 12. Only the electric field 535 is shown differently according to this exemplary embodiment. According to this exemplary embodiment, electrodes 230, 445, and 455 are driven such that another nucleated cell 515, also referred to as a non-target cell, is held in the microcavity 110. The voltages are to be selected such that contact between the non-target cell and the cavity floor is barely avoided (non-contact single cell sorting).

FIG. 14 shows a schematic diagram representation of a voltage curve 1400 according to an exemplary embodiment for a microfluidic device. The voltage curve 1400 shown in this case illustrates the behavior of a voltage U applied in the microcavity over a time t. The voltage U is applied to the microcavity. The voltage referred to as voltage U_{DEP} according to this exemplary embodiment represents, e.g., predetermined reference values representing, e.g., a minimum threshold value and a maximum threshold value. According to this exemplary embodiment, a plurality of differently applied voltage waveforms are shown, corresponding, e.g., to the operating modes as described in FIGS. 9 to 13. In other words, by adjusting and/or changing the voltage U in the individual microcavities and/or at the individual electrodes, the electric field is changed, as shown in FIGS. 9 to 13.

According to this exemplary embodiment, a first curve 1405 and a second curve 1410 represent the operating mode described in FIG. 9, in which the electrical cage is open. The first curve 1405 in this case indicates that voltage U is in this case applied to electrodes 230 and 455. The second curve 1410 illustrates that the voltage U is applied to the further electrode 445. U_{DEP,min} denotes the time average of curves 1405 and 1410.

A third curve 1415 and a fourth curve 1420 illustrate the operating mode described in FIG. 10. It is in this case clear that the third curve 1415 is implemented as a voltage variant to the first curve 1405, which is also applied to electrodes 230 and 455. Similarly, the fourth curve 1420, as a voltage variant of the second curve 1410, shows the voltage curve of the further electrode 445.

Fifth and sixth curves 1425 and 1430 further represent the operating modes shown in FIGS. 11 to 13. U_{F,Z} is in this case the time average of curve 1425, and U_{F,S} is in this case the time average of curve 1430.

In other words, a target cell can, e.g., be released according to the following:

$$0 \leq \frac{{U}_{F , s}}{{U}_{DEF , max}} = 0.3 \leq \frac{{U}_{F , z}}{{U}_{DEF , max}} = 0.6 \leq 1$$

FIG. 15 shows a schematic diagram of a microcavity 110 with dimensions according to an exemplary embodiment. The microcavity 110 is the same as or at least similar to the microcavity 110 described in one of FIG. 1, 2, or 4 to 13. According to this exemplary embodiment, the electrodes 230, 445 and 455 are further arranged concentrically. The dimensions shown in FIG. 15 are merely to be understood as examples and can deviate in alternative exemplary embodiments.

In other words, according to this exemplary embodiment, a side view of an electrified silicon technology microcavity 110 with exemplary dimensions is shown. FIG. 15 further shows a size comparison with a cell 510 located within the cavity 110 with a maximum (large dashed circle) and minimum (small circle) expected diameter.

According to this exemplary embodiment, cavity 110 comprises an exemplary diameter of 50 μm and a depth of about 37.5 μm. The numerical examples explained hereinafter refer to a trajectory of the lightest and smallest expected sphere-like CTC (ρₚ=1070 kg/m3 and R=3 μm) dielectrophoretically manipulated in a physiological medium

$$( {σ}_{m} \approx 1.4 \frac{S}{m} )$$

through electrified microcavities 110. Sufficiently large negative DEP forces (Re(\tilde{f}_{CM})≈−0.23) in physiological media can be generated, without electrolysis effects and with minimal transmembrane voltages, at operating frequencies between approximately 1 MHz to 10 MHz. The water-like wash buffer is intended to have a density ρ_{f}=1000 kg/m3 and a viscosity η=1 mPa·s.

The first part of the process makes do with comparatively small volumes of whole blood (≤20 μL) as “input”. The “output” represents an ideal starting situation for the second part of the process, a single cell sorting by DEP. At final optical detection, CTCs being isolated are expected to be among less than 80000 to 220000 leukocytes in less than 100 μL of blood lysate, making such a cell suspension interpretable as a relatively small biopsy overall.

Loading: An open DEP cage results in, e.g., an operating voltage U_{DEP}=U_{DEP}, min=1.5 V_{RMS}.

If the 50 μL chamber volume is exchanged, e.g., 30 times within 5 minutes to ensure sufficient optical transparency and purity of the chamber (i.e., if it is rinsed out with a wash buffer), then this results in a volumetric flow of 5 μL/s, which can be set on average at a constant level using a microfluidic system. For a chamber with the face input and output area of 12.5 mm×320 μm, this implies an average flow velocity of ū=ūₘₐₓ=1250 μm/s. Closed DEP cages formed at an operating voltage U_{DEP}=U_{DEP}, max=5 V_{RMS} have sufficient holding force to resist flow forces or turbulence induced by washing within cavities 110.

FIG. 16 shows a flowchart of a method 1600 according to an exemplary embodiment for trapping at least one nucleated cell using at least one electrode for a microfluidic device. This can refer to a method 1600 applicable in one of the microfluidic device setups described with reference to the preceding drawings. The method 1600 in this case comprises a step 1605 of outputting and a step 1610 of providing. During the step 1605 of outputting, an application signal is output that causes a sample liquid containing the at least one nucleated cell to be applied to a carrier substrate of the microfluidic device. During the step 1610 of providing, a current signal is provided to an interface to the at least one electrode to generate an electric field at or in a microcavity of the carrier substrate designed to trap the at least one nucleated cell as a target cell in the microcavity. According to this exemplary embodiment, during the step 1605 of outputting, the application signal is output that causes a lysate to be applied to the carrier substrate to obtain a cell sediment containing the at least one nucleated cell and a cell suspension of a lysate. In other words, e.g., a certain time period elapses in order for the cell sediment to settle. Further optionally, during or after the step 1610 of providing, a release signal is provided to the electrode after trapping the nucleated cell to release another nucleated cell from the sample liquid as a non-target cell from the electric field. The release can also relate to an optional new step 1630.

According to this exemplary embodiment, the method 1600 further comprises a step 1615 of changing an amperage and/or a voltage after the step 1605 of outputting, before or after the step 1610 of providing to change the electric field, e.g. to strengthen or weaken it. In an optional step 1620 of identifying after the step 1610 of providing, the nucleated cells are identified from the sample liquid and, in particular, optically detected and/or quantified from the cell sediment.

Further optionally, the method 1600 comprises a step 1625 of washing the sample liquid. The sample liquid is in this case washed using a wash buffer after the step of providing 1610 in order to wash a suspension of the sample liquid out of the microcavity.

The method steps presented in this case can be repeated as well as performed in a different order than described.

FIG. 17 shows a block diagram of a device 1700 according to an exemplary embodiment. The device 1700 is implemented, e.g., as a control apparatus or a control unit designed to control or perform a method for trapping at least one nucleated cell using at least one electrode for a microfluidic device, as described in FIG. 16. To this end, the device 1700 comprises an output unit 1705 for outputting an application signal 1710 that causes a sample liquid comprising the at least one nucleated cell to be applied to a carrier substrate of the microfluidic device, and a supply unit 1715 for supplying a current signal 1720 to an interface with the at least one electrode to generate an electric field at or in a microcavity of the carrier substrate designed to trap the at least one nucleated cell as a target cell in the microcavity. Further, the providing unit 1715 is designed to only optionally provide a release signal 1725 after the nucleated cell is trapped to the electrode to release another nucleated cell from the sample liquid as a non-target cell from the electric field. The device 1700 further comprises a changing unit 1730 that causes a change in an amperage to strengthen, weaken, and/or change the electric field. According to this exemplary embodiment, the device 1700 further comprises a washing unit 1735 designed to effect washing of the sample liquid using a wash buffer to wash a suspension of the sample liquid out of the microcavity. Further optionally, the device 1700 comprises an identification unit 1740 designed to identify the nucleated cells from the sample liquid, in particular to optically detect and/or quantify the nucleated cells from the cell sediment.

FIG. 18a shows a schematic diagram of an exemplary embodiment of a carrier substrate 105. The carrier substrate 105 shown in this case is the same as or at least similar to the carrier substrate 105 described in FIG. 2, which is, e.g., arranged or arrangeable in a microfluidic device 100. Such a microfluidic device 100 was previously described in at least one of FIGS. 1 to 8. According to this exemplary embodiment, the microcavities 110 are arranged in a grid-like manner or as a passive matrix, i.e., in rows and columns. Microcavities 110 are further electrically coupled to a plurality of switching units 1800, e.g., having corresponding electrical contacts. More specifically, all microcavities 110 are coupled line-by-line and column-by-column with one switch each, which further means that the microcavities 110 can also be controlled line-by-line and/or column-by-column.

According to this exemplary embodiment, a microcavity 110 centered in the carrier substrate 105 is shown as a microcavity 110 of interest. The microcavities 110 directly connected to it are in this case implemented as critical adjacent microcavities 110, each of which has a voltage drop, symbolically represented by a lightning symbol 1805.

The lightning symbol 1805 represents a parasitic electrical crosstalk and means that a rearrangement of the field distribution of the center cavity 110 of interest undesirably leads to an undefined rearrangement of the states in the neighboring microcavities 110.

In other words, the microcavities 110 are based on ground electrodes with a basically planar layer structure, which are arranged (merely by way of example) in the form of separately controllable and mutually insulated columns with point-shaped electrodes as the first, lowest metal layer and rows with ring-shaped electrodes as the second, middle metal layer. However, since a passive matrix is shown according to this exemplary embodiment, which is characterized by a simple crossover of conductive traces, a voltage drop cannot be applied or read only at a microcavity 110 of interest. On the other hand, to achieve a state of selective addressing, the passive matrix is extended by suitable integrated transistor circuits both at intersection points of traces and at the corresponding columns and rows to form an active matrix, as described and/or illustrated in FIG. 18b.

FIG. 18b shows a schematic diagram of an exemplary embodiment of a carrier substrate 105. The carrier substrate 105 shown in this case is, e.g., similar to the carrier substrate 105 described in FIG. 18a, whereby the carrier substrate 105 shown in this case is designed as an active matrix according to this exemplary embodiment. In other words, although the individual microcavities 110 are indeed also arranged in rows and columns, they are each individually coupled or couplable to a switch. However, according to this exemplary embodiment, this is shown in a simplified manner for the sake of clarity. It follows that the microcavities 110 can each be individually controlled with their own switching unit and by means of their own control signal 1850, and electrical crosstalk and thus influencing of adjacent microcavities 110 is prevented.

In other words, the microfluidic device 100 is enhanced with active circuit elements that enable “ideal” selective addressing of individual microcavities 110 of interest, such that the carrier substrate 105 is in the form of the active matrix. Microcavities 110 of interest include, e.g., microcavities 110 in which DEP manipulation has been detected and/or electrochemical detection has occurred. The active circuit elements further enable electrodes within a microcavity 110 to be used not only as manipulation elements for dielectrophoretic movement of cells, but also as sensing elements for electrochemical detection of particles delivered by cells in solution. This further extends the capabilities of an on-chip analysis after the sealing of microcavities 110, e.g. described in FIGS. 20a to 20b, in order to monitor, e.g., environmental conditions for a cell 510 in a microcavity 110.

The circuit elements of a basically planar active matrix could be implemented in the form of semiconductor integrated circuit technology. For example, complementary metal-oxide semiconductor (CMOS) technology, which is monolithically manufactured in silicon, can be used for this purpose. Alternatively, it is conceivable to use other technologies, e.g. a bipolar technology and semiconductor materials, e.g. gallium arsenide. The non-linear circuit components required for selective addressing of individual microcavities 110, meaning, e.g., transistors, memory elements, diodes, etc., can typically be fabricated off-the-shelf in any of these technologies. If an active matrix is achieved using semiconductor circuit technology, then the described full-area counter-electrode, which is also described as the third electrode, can be constructed as the third, uppermost metal layer, e.g. with the aid of photoresist systems. For this purpose, only SU-8 photoresist is used only by way of example and is spun on in a first thick layer, e.g. >30 μm, unmodified, in order to define the microcavities 110, and then in a second thin layer, e.g. <1 μm, with metal particles, e.g. silver, added in order to define the full-area counter-electrode. After what is referred to as softbaking, exposure, development and hardbaking of both layers, the counter-electrode is optionally electroplated with a chemically inert metal, e.g. gold, to increase the electrical conductivity. The electrical contacting of a semiconductor chip is achieved by, e.g., connecting bonding wires to a carrier circuit board to which a chip has previously been bonded.

If electrodes within a microcavity 110 are also to be used as sensing elements for electrochemical detection of particles in solution delivered by cells 510, then they can be functionalized beforehand with suitable counterpart particles to increase sensitivity. The counterpart particles have the task, e.g., of effectively binding the particles to be detected or enhancing the electrochemical reaction between the particle and the electrode. Sensing elements could be beneficial for numerous applications. For example, antibodies of interest (=particles) secreted by B lymphocytes could be identified in this way for drug production after the electrodes have been functionalized with the corresponding antigen (=counterpart particle). Another example is cell cultivation for cell line development, where the electrodes as sensing elements are used to precisely track and control growth conditions, e.g., pH level, O₂ or CO₂ content, and/or glucose concentration, in real time.

FIG. 19a shows a schematic cross-sectional view of an exemplary embodiment of a microfluidic device 100. According to this exemplary embodiment, a nucleated cell 510 is in this case arranged in each of the microcavities 110. The lysate is in this case arranged as an aqueous medium around the cells 510 and in an intermediate region 1900 between the carrier substrate 105 and a transparent cover 1905. For example, the intermediate region 1900 can also be referred to as the detection region of the microfluidic device 100. According to this exemplary embodiment, each of the microcavities 110 comprises a first electrode 1910 and a second electrode 1915. The electrodes 1910, 1915 can also be referred to as ground electrodes and are designed to generate, e.g., an electric field and/or a magnetic field in an operating state. The generated fields keep the nucleated cells 510 in the respective microcavities 110 while, e.g., the lysate is washed. In this case, the first electrode 1910 is implemented centrally and in a point-like manner on a cavity floor 1920, and the second electrode 1915 is arranged, e.g., in a ring-like manner around the first electrode 1910. The electrodes 1910, 1915 do not touch each other. According to this exemplary embodiment, the first electrode 1910 is designed as a positive terminal, and the second electrode 1915 is designed as a negative terminal.

According to this exemplary embodiment, the carrier substrate 105 further comprises a third electrode 1925 arranged in a planar manner on a substrate surface in accordance with this exemplary embodiment. The third electrode 1925 is also designed as a positive pole and follows the voltage curve over time shown in FIG. 14.

In other words, the microcavities 110 are shown as electrified microcavities 110, each with a loaded nucleated cell 510.

FIG. 19b shows a schematic cross-sectional view of an exemplary embodiment of a microfluidic device 100. The microfluidic device 100 illustrated in this case is, e.g., similar to the microfluidic device 100 described in FIG. 19a. According to this exemplary embodiment, only particles 1950 from each of the nucleated cells 510 are arranged in a dome-like manner around the corresponding cell 510 such that they each affect an adjacent microcavity 110. In other words, only exemplary electrical and/or microfluidic crosstalk is shown in this case.

FIG. 20a shows a schematic cross-sectional view of an exemplary embodiment of a microfluidic device 100. The microfluidic device 100 illustrated in this case is, e.g., similar to the microfluidic device 100 described in FIG. 19a. A nucleated cell 510 is in this case also arranged in each of the microcavities 110 and held in position by electrodes 1910, 1915, 1925. However, according to this exemplary embodiment, the microfluidic device 100 is exposed to a non-aqueous medium 2000 or a non-aqueous phase comprising, e.g., a silicone oil or air in order to eliminate microfluidic crosstalk of interacting or adjacent, microcavities 110, as described in, e.g., FIG. 19b, and to seal the microcavities 110. In other words, the microcavities 110 are sealed given the choice of non-aqueous medium 2000 and associated physical properties.

According to this exemplary embodiment, during the single cell analysis on-chip, the chip, i.e., the microfluidic device 100 is flushed with at least one medium 2000 non-aqueous phase. This achieves sealing and thus isolation of cells 510 in aqueous phase, which is described in this case as an aqueous medium with physiological properties, in microcavities 110.

In other words, the lysate, meaning the aqueous phase layer, above the cavities 110 is first displaced by a non-aqueous phase medium 2000 after a loading of the cells 510 in order to achieve aqueous phase sealing of the cells 510 within microcavities 110 for single cell analyses on-chip. If individual cells 510 are additionally intended to be output off-chip into collection vessels for analyses, then the previously input non-aqueous phase 2000 is displaced again with an aqueous phase, returning the situation to its initial state.

FIG. 20b shows a schematic cross-sectional view of an exemplary embodiment of a detection chamber of a microfluidic device 100. The detection chamber shown in this case corresponds to the detection chamber described in FIG. 20a. According to this exemplary embodiment, the intermediate region 1900 is only completely filled with the non-aqueous medium 2000 except for the microcavities 110, so that the microcavities 110 are isolated from each other. In other words, aqueous medium is only present in the microcavities 110 themselves.

In other words, an elimination of parasitic microfluidic crosstalk is shown. The electrified microcavities 110 with nucleated cells 510 in aqueous phase are completely insulated from each other by the non-aqueous medium 2000. Diffusive and convective carryover of particles delivered by cells 510 into solution is in this case no longer possible, and analyses in individual microcavities 110 take place independently.

If an exemplary embodiment comprises an “and/or” conjunction between a first feature and a second feature, then this is to be understood such that the exemplary embodiment according to one embodiment comprises both the first feature and the second feature and, according to a further embodiment, comprises either only the first feature or only the second feature.

1. A method for trapping at least one nucleated cell using at least one electrode for a microfluidic device, comprising:
outputting an application signal that causes a sample liquid comprising the at least one nucleated cell to be applied to a carrier substrate of the microfluidic device; and
providing a current signal to an interface with the at least one electrode in order to generate, at or in a microcavity of the carrier substrate, an electric field configured to trap the at least one nucleated cell as a target cell in the microcavity.

2. The method according to claim 1, further comprising:
varying an amperage in order to change the electric field so as to strengthen or weaken the field after the step of outputting or before or after the step of providing, wherein the electric field is established and/or varied between the electrode and a counter-electrode arranged opposite the electrode in or at the microcavity.

3. The method according to claim 1, wherein during the step of providing, the current signal is output to interface with the at least one electrode and with at least one other electrode arranged in an adjacent microcavity such that a different electric field is generated at the at least one other electrode than at the electrode, wherein the field generated at the electric electrode differs with respect to a direction and/or intensity from the electric field generated at the other electrode and/or wherein the other electric field is generated at the other electrode, which is arranged in a microcavity that is arranged in a common column or a common row with respect to the microcavity having the electrode.

4. The method according to claim 1, further comprising:
washing the sample liquid using a wash buffer after the step of providing in order to wash a suspension of the sample liquid out of the microcavity, and/or wherein, during or after the step of providing, a release signal is provided to the electrode after trapping the nucleated cell in order to release another nucleated cell from the sample liquid as a non-target cell from the electric field.

5. The method according to claim 1, wherein, during the step of outputting, the application signal is output, thus causing a lysate to be applied to the carrier substrate in order to obtain a cell sediment with the at least one nucleated cell and a cell suspension of a lysate and/or wherein a step of identifying the nucleated cells from the sample liquid is provided after the step of providing, wherein, during the step of identifying, the nucleated cells from a cell sediment are optically detected and/or quantified.

6. The method according to claim 1, wherein, during the step of providing, the nucleated cell or at least one further nucleated cell is trapped in a trapping plane of the microcavity and/or wherein, by way of the release signal, the cell or at least one further nucleated cell is released from the sample liquid from the electric field into a transport plane.

7. A method for trapping at least one nucleated cell using at least one electrode for a microfluidic device, comprising:
applying a sample liquid comprising the at least one nucleated cell to a carrier substrate of the microfluidic device; and
generating an electric field at or in a microcavity of the carrier substrate having the at least one electrode, which field is configured to trap the at least one nucleated cell as a target cell in the microcavity.

8. A device designed to perform and/or control the steps of the method according to claim 1 in respective units.

9. A computer program configured to perform and/or control the steps of the method according to claim 1.

10. A machine-readable storage medium on which the computer program according to claim 9 is stored.

11. A microfluidic device for trapping at least one nucleated cell in a sample liquid, wherein the microfluidic device is designed as a lab-on-chip cartridge, wherein the microfluidic device comprises:
a carrier substrate configured to contain the sample liquid, wherein the carrier substrate comprises at least one microcavity; and
at least one electrode arranged on or in the microcavity in order to generate an electric field configured to trap the nucleated cell in the microcavity.

12. The microfluidic device according to claim 11, wherein the carrier substrate comprises a plurality of microcavities, each having at least one electrode, wherein the microcavities are arranged in a matrix-like manner on the carrier substrate.

13. The microfluidic device according to claim 12, wherein the at least one electrode is arranged on a cavity floor and/or in a cavity wall of at least one of the microcavities.

14. The microfluidic device according to claim 12, wherein the electrodes in each of the microcavities are individually controllable, and/or wherein the electrode is annular, point-like, and/or layer-like in shape, wherein a control unit is further provided which is designed to impress a mutually independent voltage on each of the electrodes in the different microcavities.

15. The microfluidic device according to claim 11, wherein the microcavity comprises at least one further electrode, wherein the electrode and the at least one further electrode are electrically insulated from each other.

16. The microfluidic device according to claim 11, wherein at least one counter-electrode is arranged at the microcavity, wherein the counter-electrode is arranged opposite the electrode and/or the at least one further electrode in or at the microcavity and/or is electrically insulated from the electrode and/or the at least one further electrode.
